(19) United States
(12) Patent Application Publication
(10) Pub. No.: US 2026/0365591 A1
(43) Pub. Date: Nov. 19, 2026

(54) INCREASED PROCESSING FLEXIBILITY IN GASIFICATION

(71) Applicant: SunGas Renewables, Inc., Houston, TX (US)

(72) Inventors: Clifton G. KEELER, Spring, TX (US); Sekar DARUJATI, Houston, TX (US)

(21) Appl. No.: 19/130,190

(22) PCT Filed: Nov. 15, 2023
(86) PCT No.: PCT/US2023/079738

§ 371 Date: May 15, 2025

Related U.S. Application Data
(60) Provisional application No. 63/425,786, filed on Nov. 16, 2022.

(51) Int. Cl. C07C 1/04; C07C 29/151; C10J 3/00

(52) U.S. Cl. CPC C07C 1/0495; C07C 29/1518; C10J 3/00

(57) ABSTRACT

Gasification processes utilizing carbonaceous feeds and preferably biomass are disclosed, which can implement one or more strategies for tar and ash management, heat and/or steam integration, as well as byproduct utilization, to address a number of challenges that can often involve conflicting requirements in terms of conditions (particularly temperature) and compositions. For example, a combination of direct and indirect cooling, together with other equipment, can avoid the need for a radiant syngas cooler to mitigate ash deposition. Alternatively, or in combination, available process heat and process steam may be exploited to attain acceptable temperature and moisture levels in the syngas being subjected to water-gas shift. Yet other advantages arise from the direct processing of material from a gaseous or liquid byproduct of downstream conversion or separation, via combustion and/or conversion, to improve heat integration and/or carbon utilization. Specific approaches utilize residence time variation, integration of generated steam, cross-exchanging of heat, utilization of byproducts, and/or recycle of treated water, in the production of synthesis gas or its downstream conversion products (e.g., hydrocarbons or methanol), for improved processing flexibility and/or economics.

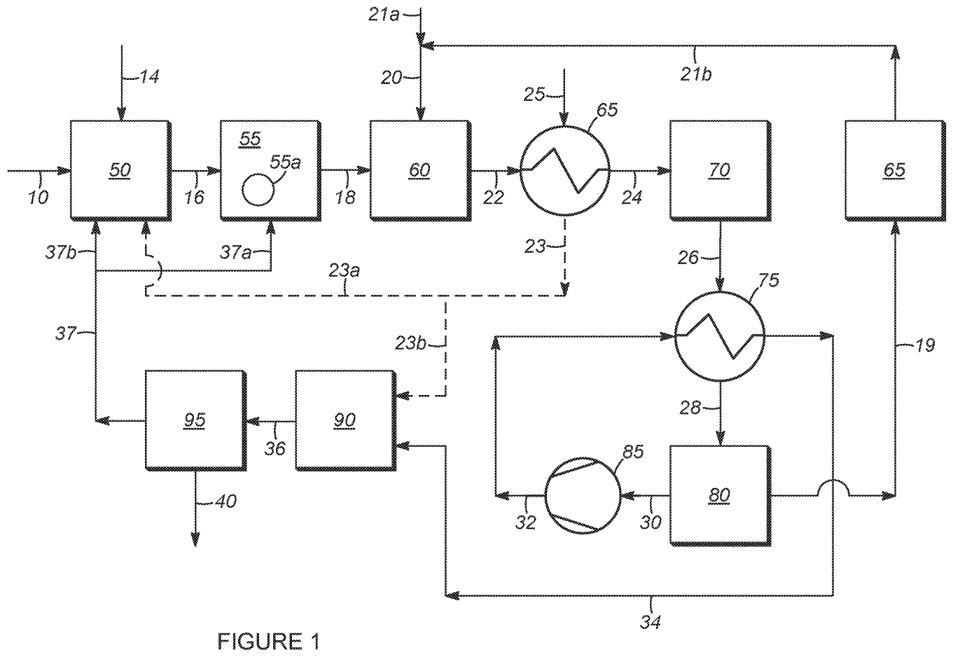

### CROSS-REFERENCE TO RELATED APPLICATIONS

This application claims priority to U.S. Provisional Application No. 63/425,786, filed Nov. 16, 2022, the content of which is hereby incorporated by reference in its entirety.

### FIELD OF THE INVENTION

Aspects of the invention relate to gasification processes in which processing flexibility and/or efficiency are enhanced through a number of possible operating strategies, including management of temperature/residence time for tar conversion, heat integration, and utilization of byproducts obtained from downstream conversion and separation of syngas produced from gasification.

### DESCRIPTION OF RELATED ART

The gasification of coal has been performed industrially for over a century in the production of synthesis gas (syngas) that can be further processed into transportation fuels. More recent efforts toward developing energy independence with reduced greenhouse gas emissions have led to a strong interest in using biomass as a gasification feed, and thereby an alternative potential source of synthesis gas, as well as its downstream conversion products. Generally, biomass gasification is performed by partial oxidation in the presence of a suitable oxidizing gas containing oxygen and other possible components such as steam. Gasification at elevated temperature and pressure, optionally in the presence of a catalytic material, produces an effluent with hydrogen and oxides of carbon (CO, CO₂), as well as hydrocarbons such as methane. This effluent, which is often referred to as synthesis gas in view of its H₂ and CO content, must be cooled significantly and also treated to remove a number of undesired components that can include particulates, alkali metals, halides, and sulfur compounds, in addition to byproducts of gasification that are generally referred to as tars and oils. Furthermore, downstream conversion of the synthesis gas to value-added products often requires its hydrogen content to be increased, relative to that obtained from gasification alone.

The economics of biomass gasification and the effective utilization of the produced synthesis gas for obtaining desired end products are impacted by a number of complex and interacting processing objectives, as well as the associated equipment requirements. For example, undesired tar components in the gasifier effluent, which can include fused ring molecules such as naphthalene and pyrene, pose significant challenges in terms of the tendency of such high boiling-temperature molecules to condense from the vapor phase onto lower-temperature surfaces encountered downstream of the gasifier. Physical deposition of tars and oils is known to cause fouling/clogging of process lines, valves, reactors, and other equipment. For these reasons, the thermal destruction of tar is commonly practiced, but this, in turn, requires temperatures that are sufficient to cause melting or slagging of ash that is also present in the synthesis gas obtained from biomass gasification. The “sticky” ash, like the tar itself, is known to deposit on and plug downstream equipment operating at cooler temperatures, including apparatuses used for upgrading of synthesis gas to end products. A radiant syngas cooler (RSC) with the capability to process and remove molten byproducts is seen as a potential way to address this problem, but only at considerable cost.

Regarding the need to increase the H₂:CO molar ratio of the synthesis gas for its subsequent use in a number of reactions, the exothermic water-gas shift (WGS) reaction gas according to:
CO+H₂O↔H₂+CO₂
   is widely exploited for this purpose. The thermodynamics of this reaction govern an equilibrium shift toward hydrogen production at lower temperatures, which are generally unfavorable from the standpoint of reaction kinetics. Another factor impacting performance is the concentration of steam in the WGS reaction feed, which directionally favors the intended H₂ production. However, operations conducted to purify the gasifier effluent, or synthesis gas, in preparation for the catalytic WGS reaction, can lead to substantial cooling of this stream and/or cause its dehydration, such that process efficiency must be sacrificed to thereafter “restore” conditions, as needed to achieve acceptable conversion levels and associated hydrogen concentrations. This is practiced, for example, when gasifier-produced synthesis gas, having been treated to remove tars and oils at elevated temperatures, is then scrubbed to remove water- and water-soluble contaminants, causing significant reductions in its heat and moisture content.

A number of technical challenges therefore exist with respect to requirements for heating, cooling, removal of tar and other contaminants, and H₂:CO molar ratio adjustment, as necessary to obtain a synthesis gas product suitable for downstream conversion to higher value products. These include liquid hydrocarbons and/or oxygenates (e.g., alcohols) having varying carbon numbers, as produced according to the Fischer-Tropsch synthesis reaction. These also include methanol, produced via catalytic methanol synthesis, as well as renewable natural gas (RNG) or biomethane, produced via catalytic methanation. The conversion of synthesis gas according to these and other reactions generally also provides gaseous and/or liquid byproduct streams that can become sources of inefficiency due to yield loss. The present state of the art would benefit from improvements in flexibility and/or efficiency to alleviate constraints imposed by these and other considerations, which often involve competing processing objectives.

### SUMMARY OF THE INVENTION

Aspects of the invention are associated with the discovery of gasification processes utilizing carbonaceous feeds and preferably biomass, which can implement one or more strategies for tar and ash management, heat and/or steam integration, as well as byproduct utilization, to address a number of challenges in the art, including those described above.

More specific aspects relate to addressing effects associated with the high reactivity of biomass compared to coal, associated with an approximately 300 °C reduction in gasification temperature, toward a typical range of 750 °C to 1050 °C. This lower gasification temperature, coupled with the high content of volatile components in biomass, results in the significant production of tar that includes naphthalene and pyrene as noted above, but more generally molecules having two or more carbon atoms (e.g., C₂⁺ hydrocarbons), any of which can result in plugging problems (either directly or through further reaction to form higher molecular weight byproducts) downstream of the gasifier, upon cooling. In addition, steps undertaken to thermally destroy this tar require temperatures of about 1300 °C, well exceeding those of the gasifier and sufficient to cause melting and/or slagging of ash that is also present in tar-laden syngas stream or gasifier effluent. The molten material or slag is itself a source of potential equipment fouling and plugging, due to deposition at cooler downstream temperatures. The use of a sufficiently large-sized radiant syngas cooler (RSC) is viewed as a possible way to separate slag via a quench chamber at the bottom of this apparatus, but only at considerable expense.

To address these effects and related problems, namely tar and ash plugging/deposition, while possibly avoiding the expense of an RSC, temperature and/or residence time in a tar removal operation following gasification can be effectively managed. This can “tailor” the tar removal operation to the existing state of the gasifier effluent or even the particular carbonaceous feed, advantageously allowing for a reduction in temperature (or peak temperature) to which ash, also present in the gasifier effluent, such as the raw gasifier effluent, is subjected while still achieving an effective degree of tar remediation as needed. The ability to increase tar conversion residence time can make such temperature reduction possible, if tar breakthrough remains absent or otherwise at an acceptably low level. In exemplary processes, the thermal reforming of tar may be performed directly following the gasifier, i.e., on the gasifier effluent, to produce additional syngas, with a particular tar removal operation utilizing a hot oxygen burner (HOB) in a partial oxidation (Pox) reactor. Downstream of, or as a part of, this reactor, or other reactor of a tar removal operation, a tar conversion residence vessel (TCRV) having additional capacity may be employed for providing additional residence time (e.g., 15 to 60 seconds) as needed for complete or substantially complete destruction (conversion) of tar, prior to subsequent cooling of the gasifier effluent. Advantageously, this additional, TCRV-mediated residence time effectively incorporates a “knob” into a reactor used for tar removal, allowing it to operate at variable temperature (e.g., 950 °C to 1350 °C), as governed by the overall temperature/residence time profile and the ability of these tar conversion conditions to reform tar to the desired extent, producing H₂, CO, and CO₂.

Without this ability to vary residence time, the normal effect of lowering temperature is simply an increase in the “slip” of tars, evidenced by an increase in any number of C₂⁺ components, residing in the tar-depleted gasifier effluent, exiting the tar removal (e.g., tar reforming) operation. The use of an effective temperature/residence time combination as described herein, however, provides additional operating flexibility for establishing conditions suitable for a particular slate of tar components and amounts, as obtained from a particular operation of the gasifier, processing a particular carbonaceous feed. Residence time variation also provides the ability to manage (establish and maintain) “minimum severity” conditions that achieve the desired reduction of tar with the least generation of undesired soft “sticky” ash and slag. To the extent that such conditions minimize the temperature (or peak temperature) used in a given tar removal operation such as involving Pox, this has the further beneficial effect of increasing or even maximizing the H₂:CO molar ratio of the tar-depleted gasifier effluent exiting this operation, in view of the WGS reaction thermodynamics. The performance requirements of a subsequent, dedicated WGS operation are thereby reduced, potentially further improving overall process economics.

Downstream of the tar reforming operation, such as directly following a TCRV of this operation, the tar-depleted gasifier effluent may be subjected to quenching, for example by the injection of water through nozzles used in a quenching operation, which may be, more particularly, a partial dry quench (PDQ) operation. Quenching may effectively cool the resulting quenched gasifier effluent to below the softening temperature of the ash, for example to a temperature in the range of 600 °C to 750 °C at which the ash is no longer “sticky.” The quenching operation can facilitate the subsequent use of a convective syngas cooler (CSC) that is generally significantly less expensive than (and may be only 20-50% of the cost of) an RSC. Moreover, while smaller and cheaper than an RSC, a CSC can generate sufficient high-pressure steam, in many cases, to satisfy the steam demands of the gasifier or WGS, and preferably both of these operations. In this regard, particular aspects of the invention relate to the addition of first and/or second portions of CSC-generated steam, respectively, to the gasifier and/or the WGS operation.

Further specific aspects relate to efficiently establishing conditions of temperature and moisture level (humidity) of syngas, such as a scrubbed gasifier effluent, being fed to the water-gas shift (WGS) reactor used for increasing hydrogen content and therefore the H₂:CO molar ratio. In this regard, following the initial cooling of the syngas obtained from gasification, filtration is normally performed to remove particulates such as fine ash, unconverted biomass, and/or condensed carbon. At this stage, the temperature and moisture level are normally favorable for introduction of the filtered syngas (e.g., filtered gasifier effluent) to the WGS reactor, but this syngas nonetheless typically contains trace contaminants such as chlorides that act as poisons of catalysts used for the WGS reaction. For example, depending on the level of chloride initially present in the carbonaceous feed (e.g., biomass), the content of chloride from compounds such as HCl in the filtered syngas (e.g., filtered gasifier effluent) can exceed 50 ppm, whereas WGS catalysts typically require the concentration of this contaminant to be less than 1 ppm or even less than 0.1 ppm in the case of certain “sweet shift” catalysts that operate under essentially sulfur-free conditions. Chlorides and other water-soluble contaminants can be removed via a scrubbing operation, such as a wet scrubber using an aqueous scrubbing medium. However, since biomass gasifiers operate at relatively low pressures (e.g., less than 15 bar) that impact the pressure used for downstream scrubbing, the scrubbed gasifier effluent, having been purified of water-soluble contaminants, typically has a temperature (e.g., less than 65 °C) and moisture content (e.g., less than 5 mol-%) that render this gas unsuitable in these respects as a feed for the WGS reactor, which typically requires an inlet temperature in the range of 225 °C to 325 °C and a moisture content exceeding 40 mol-%.

To address such challenges with respect to efficiently obtaining a syngas from gasification (e.g., biomass gasification in particular) and having suitable temperature, composition (e.g., moisture content), and purity (e.g., chloride and/or sulfur content) for effective use as a feed to a WGS operation, the gasifier effluent exiting the scrubbing operation (e.g., wet scrubber) may be heat-exchanged against a suitable process stream obtained upstream of this operation. For example, cross-exchange may be performed between the scrubbed gasifier effluent or a portion thereof and syngas exiting the CSC (e.g., as a cooled gasifier effluent, but nonetheless having an elevated temperature and corresponding heat content), and optionally following a filtration operation. This cross-exchange may thereby increase the temperature of the scrubbed product (e.g., as a cross-exchanger heated effluent) for introduction to the WGS operation with a reduced requirement for, and preferably without any, additional heat input upstream of this operation. Further advantageous process integration with the WGS operation may involve adding steam to this operation, as generated from cooling of the gasifier effluent (e.g., an un-scrubbed gasifier effluent upstream of the scrubbing operation). Any source of such steam, for example as generated in the CSC, may be added to the syngas entering the WGS operation or directly to a reactor of this operation, thereby beneficially increasing moisture content upstream of, and/or within, this operation.

Yet other more specific aspects relate to the advantageous integration of process streams generated in the downstream conversion and/or separation of synthesis gas (e.g., obtained as a product of the WGS reaction) such as to produce a renewable liquid conversion product (e.g., liquid hydrocarbons or methanol) or a renewable gaseous product (e.g., renewable natural gas (RNG) or renewable hydrogen). For example, the conversion of syngas to Fischer-Tropsch (FT) liquid hydrocarbons and alcohols generally results in the formation of a gaseous byproduct stream (“gaseous conversion byproduct”) that is normally combusted to recover, from its moderate heating value, some external process heat. As a further example, the separation of syngas to obtain purified hydrogen generally results in the formation of a gaseous byproduct stream (“gaseous separation byproduct”), such as in the case of separation by pressure swing adsorption (PSA) that generates, in addition to high purity hydrogen, a tail gas that is enriched in non-hydrogen components of syngas (e.g., CO, CO₂, H₂O, and possibly methane). Combustion of these and other gaseous byproducts, as well as liquid byproducts, of downstream conversion/separation of syngas, however, represents a low utilization of their energy and carbon content.

Enhanced integration and efficiency results from usage of gaseous and liquid byproducts generated from syngas conversion and separation operations (e.g., occurring downstream of a WGS operation) as a fuel for direct heating within a tar removal operation (e.g., the HOB) and/or as a feed to the process itself. The recovery of such byproducts for purposes within the gasification process, rather than as a source of indirect heat, may involve combusting at least a portion (e.g., a first portion) of a given gaseous or liquid byproduct, of a downstream conversion or separation, in the HOB and/or feeding at least a portion (e.g., a second portion beyond the fuel requirement of the HOB) to the gasifier, such as to the freeboard of the gasification reactor and/or within a fluidized particle bed of such reactor. In this manner, direct introduction of one or more byproducts of a syngas conversion operation or syngas separation operation can provide additional syngas (optionally in conjunction with a suitable adjustment of the process oxygen requirement), thereby increasing its overall yield, in addition to the overall carbon recovery in both the syngas as well as in the renewable syngas conversion product or renewable syngas separation product itself.

Advantages according to these and other aspects of the invention may therefore be realized over technologies according to which gasifier effluent tar is reformed at temperatures of about 1300 °C or more, exposing ash in the syngas to softening or slag formation conditions. According to process configurations described herein, such as those utilizing both direct and indirect cooling (e.g., a dry quenching operation, such as a PDQ, in conjunction with a CSC), an expensive RSC, conventionally required to mitigate ash deposition in the steam generator of this apparatus, may be avoided, i.e., an RSC may be absent in representative processes. Further advantages may be attained in view of addressing problems relating to the low temperature and moisture content of syngas exiting the scrubber in certain processes that rely on external sources of heat and/or steam addition to provide acceptable WGS characteristics. Yet other advantages may arise from the direct addition to the process of material from a gaseous or liquid byproduct of a syngas conversion operation or syngas separation operation, for combustion and/or conversion, to improve heat integration and/or carbon utilization.

Particular embodiments of the invention therefore relate to processes for the gasification of a carbonaceous feed, such as biomass that can include for example wood (e.g., wood waste) in various forms (e.g., wood chips or wood pellets), municipal solid waste (MSW), plastics (e.g., plastic waste), and other waste materials (e.g., agricultural waste), which processes benefit from increased processing flexibility and/or process stream management, in various respects as described herein. Gasification may be followed by downstream conversion and/or separation of the generated syngas to produce renewable fuels, including liquid hydrocarbons (e.g., sustainable aviation fuel or RNG) or methanol (e.g., for marine fuel), or otherwise to produce renewable hydrogen. Representative processes can utilize the thermal conversion of gasifier effluent tar with the mitigation of ash deposition; the recovery of steam, in a CSC, for use in the gasifier and/or a WGS operation; the recovery of heat from the gasifier and/or downstream tar removal operation, for input into the scrubbed gasifier effluent prior to a WGS operation; and/or the recycle, to the process (e.g., the gasifier or tar removal operation), of least a portion of a gaseous or liquid byproduct generated in the downstream conversion of syngas or downstream separation of syngas.

These and other embodiments, aspects, and advantages relating to the present invention are apparent from the following Detailed Description.

### BRIEF DESCRIPTION OF THE DRAWING

A more complete understanding of the exemplary embodiments of the present invention and the advantages thereof may be acquired by referring to the following description in conjunction with the accompanying Figure.

The FIGURE depicts a flowscheme illustrating an embodiment of a process for the gasification of a carbonaceous feed, which process employs a number of possible aspects as described herein for improving process flexibility, such as through tar and ash management, heat and/or steam integration, and/or utilization of byproducts (e.g., tail gas and/or sour water). For the sake of simplicity, multiple features are illustrated and described in the single FIGURE, with the understanding that not all features (e.g., not all individual operations and their associated process streams and equipment) are required and that various specific features, such as residence time variation, integration of generated steam, cross-exchanging heat, utilization of byproducts, and recycle of treated water, can be implemented independently of others.

In order to facilitate explanation and understanding, the FIGURE provides an overview of these and other features for implementation in gasification processes. Some associated equipment such as certain vessels, heat exchangers, valves, instrumentation, and utilities, are not shown, as their specific description is not essential to the implementation or understanding of the various aspects of the invention. Such equipment would be readily apparent to those skilled in the art, having knowledge of the present disclosure. Other processes for producing syngas and/or its conversion products such as renewable liquids, according to other embodiments within the scope of the invention and having configurations and constituents determined, in part, according to particular processing objectives, would likewise be apparent.

### DETAILED DESCRIPTION

The expressions “wt-%” and “mol-%,” are used herein to designate weight percentages and molar percentages, respectively. The expressions “wt-ppm” and “mol-ppm” designate weight and molar parts per million, respectively. For ideal gases, “mol-%” and “mol-ppm” are equal to percentages by volume and parts per million by volume, respectively.

The term “substantially,” as used herein, refers to an extent of at least 95%. For example, the phrase “substantially all” may be replaced by “at least 95%.” The phrases “all or a portion” or “at least a portion” are meant to encompass, in certain embodiments, “at least 50% of,” “at least 75% of,” “at least 90% of,” and, in preferred embodiments, “all.” Likewise, designated portions, such as a “first portion” or “second portion” may represent these percentages (but not all) of the total, and particularly these percentages (but not all) of the total process stream to which they refer.

Reference to any starting material, intermediate product, or final product, which are all preferably process streams in the case of continuous processes, should be understood to mean “all or a portion” of such starting material, intermediate product, or final product, in view of the possibility that some portions may not be used, such as due to sampling, purging, diversion for other purposes, mechanical losses, etc. Therefore, for example, the phrase “subjecting the gasifier effluent to a tar removal operation” should be understood to mean “subjecting all or a portion of the gasifier effluent to a tar removal operation.” Even in the case of “all or portion” being the understood meaning, this phrase when expressly stated nonetheless encompasses certain and preferred embodiments as noted above.

Representative processes described herein for the gasification of a carbonaceous feed may comprise a number of unit operations, with one of such operations stated as being performed or carried out “before,” “prior to,” or “upstream of” another of such operations, or with one of such operations being performed or carried out “after,” “subsequent to,” or “downstream of,” another of such operations. These quoted phrases, which refer to the order in which one operation is performed or carried out relative to another, are in reference to the overall process flow, as would be appreciated by one skilled in the art having knowledge of the present specification. More specifically, the overall process flow can be defined by the bulk gasifier effluent flow, including bulk flows of both the un-scrubbed gasifier effluent and scrubbed gasifier effluent, as well as the bulk WGS product flow, as such flow(s) is/are subjected to operations as defined herein. Insofar as the quoted phrases are used to designate order, in specific embodiments these phrases mean that one operation immediately precedes or follows another operation, whereas more generally these phrases do not preclude the possibility of intervening operations. Therefore, for example, the phrase “ . . . WGS operation . . . downstream of the tar removal operation . . . ” means, according to a specific embodiment, that the water-gas shift (WGS) operation immediately follows the tar removal operation. However, this phrase more generally, and preferably, means that one or more intervening operations can be performed or carried out between these operations (e.g., a quenching operation, a convective syngas cooler (CSC), a filtration operation, cross-exchanging heat, a scrubbing operation, and compression, according to the embodiment illustrated in the FIGURE). Therefore, to the extent that representative processes described herein are defined as including certain unit operations, unless otherwise stated or designated (e.g., by using the phrase “consisting of”), such processes do not preclude the use of other operations, whether or not specifically described herein.

Specific processes described herein are defined by a gasifier, a scrubbing operation (e.g., wet scrubber) downstream of the gasifier, and a WGS operation downstream of the scrubbing operation. The gasifier provides a “gasifier effluent” and the WGS operation provides a “WGS product.” The term “gasifier effluent” is a general term that refers to the effluent of the gasifier, whether or not having been subjected to one or more operations downstream of the gasifier and upstream of the WGS operation. The “gasifier effluent” may be more particularly designated as an “un-scrubbed gasifier effluent” or a “scrubbed gasifier effluent,” which are also general terms but add specificity in terms of characterizing the gasifier effluent depending on whether or not it has been subjected to a scrubbing operation.

The terms “gasifier effluent” and “un-scrubbed gasifier effluent” encompass more specific terms that designate (i) the effluent provided directly by the gasifier, i.e., the “raw gasifier effluent,” (ii) the raw gasifier effluent having been subjected to at least a tar removal operation, i.e., a “tar-depleted gasifier effluent,” having a lower concentration of tars and oils relative to the raw gasifier effluent, (iii) the raw gasifier effluent having been subjected to at least a dry quenching operation, i.e., a “quenched gasifier effluent,” having a lower temperature and higher moisture (H₂O) concentration relative to the raw gasifier effluent, resulting from direct quenching (e.g., partial quenching) with water, (iv) the raw gasifier effluent having been subjected to at least a CSC, i.e., a “cooled gasifier effluent” having a lower temperature relative to the raw gasifier effluent, resulting from heat transfer for external steam generation, (v) the raw gasifier effluent having been subjected to at least a filtration operation, i.e., a “filtered gasifier effluent,” having a lower solid particle content relative to the raw gasifier effluent, (vi) the raw gasifier effluent having been subjected to cross-exchanging of heat, i.e., a “cross-exchanger cooled effluent,” having a lower temperature relative to the raw gasifier effluent, resulting from heat transfer with at least a portion of material removed from the scrubbing operation, and (vii) the raw gasifier effluent having been subjected to any other operation upstream of the scrubbing operation, whether or not specifically described herein.

Likewise, the terms “gasifier effluent” and “scrubbed gasifier effluent” encompass more specific terms that designate (viii) the raw gasifier effluent or un-scrubbed gasifier effluent having been subjected to a scrubbing operation to reduce its content of water-soluble contaminants (e.g., chlorides), (ix) the raw gasifier effluent or scrubbed gasifier effluent having been subjected to compression, i.e., a “compressed, scrubbed gasifier effluent,” having a higher pressure relative to the scrubbed gasifier effluent, (xi) the raw gasifier effluent or scrubbed gasifier effluent having being subjected to the cross-exchanging of heat, i.e., a “cross-exchanger heated effluent,” having a higher temperature relative to the scrubbed gasifier effluent, resulting from heat transfer with at least a portion of the un-scrubbed gasifier effluent, such as at least a portion of material removed from the filtration operation, and (xii) the raw gasifier effluent or scrubbed gasifier effluent having been subjected to any other operation downstream of the scrubbing operation, whether or not specifically described herein.

With respect to the cross-exchanging of heat, such as in the case of utilizing a gasifier effluent cross-exchanger that is positioned upstream of the scrubbing operation, the cross-exchanger heated feed and the cross-exchanger cooled effluent provide examples of the gasifier effluent, according to particular embodiments, that may be characterized as an un-scrubbed gasifier effluent. The cross-exchanger cooled feed and the cross-exchanger heated effluent provide examples of the gasifier effluent, according to particular embodiments, that may be characterized as a scrubbed gasifier effluent. According to specific embodiments, such as the embodiment illustrated in the FIGURE, (a) the cross-exchanger heated feed may comprise all or a portion of the filtered gasifier effluent, (b) the cross-exchanger cooled feed may comprise all or a portion of the compressed, scrubbed gasifier effluent, and/or (c) the cross-exchanger heated effluent may comprise all or a portion of a feed to the WGS operation (e.g., to which a source of steam may be added, prior to this operation).

The terms “gasifier effluent,” “un-scrubbed gasifier effluent,” and “scrubbed gasifier effluent,” and any of the more specific examples (i)-(xii) of these terms, encompass products (e.g., flow streams) that are upstream of, and optionally may be fed to, the WGS operation.

The term “WGS product” is a general term that refers to a product of the WGS operation, all or a portion of which may, according to particular embodiments, be fed to a syngas conversion operation or a syngas separation operation to provide as a value-added product, a renewable syngas conversion product or a renewable syngas separation product. The term “WGS product” encompasses all or a portion of the product provided directly by the WGS operation, or otherwise such product after having been subjected to heating, cooling, pressurization, depressurization, and/or purification, such as acid gas removal. The term “syngas,” or alternatively “synthesis gas product,” insofar as they relate to streams comprising H₂ and CO, are used herein to generally refer to the gasifier effluent, whether an un-scrubbed gasifier effluent or a scrubbed gasifier effluent as defined above, or the WGS product.

Particular examples of renewable syngas conversion products and renewable syngas separation products include both renewable liquid products (e.g., liquid hydrocarbons or methanol) and renewable gaseous products (e.g., renewable natural gas (RNG) or renewable hydrogen). The modifiers “syngas conversion” and “syngas separation,” as well as the modifiers “conversion” and “separation,” as used in the terms “renewable syngas conversion product,” “renewable syngas separation product,” “gaseous conversion byproduct,” “liquid conversion byproduct,” and “gaseous separation byproduct” are meant to more specifically designate the origin of these products and byproducts, as being obtained from either a syngas conversion operation (e.g., comprising a Fischer-Tropsch reaction stage, a methanol synthesis reaction stage, or a methanation reaction stage) or a syngas separation operation (e.g., comprising a hydrogen purification stage, such as in the case of syngas separation by pressure swing adsorption (PSA) and/or the use of a membrane). Any such syngas conversion operation or syngas separation operation is preferably performed on the WGS product that can yield an increased, and more favorable, H₂:CO molar ratio, in terms of efficiently performing the desired conversion or separation. The use of the modifiers “separation” and “conversion” in the terms noted above to modify products and byproducts does not preclude such products and byproducts being obtained from a combination of separation and conversion.

In achieving various objectives and associated advantages as described herein, particular embodiments of the invention are directed to processes for gasification of a carbonaceous feed, in order to produce a synthesis gas product and/or optionally a downstream renewable syngas conversion product (e.g., liquid hydrocarbons or methanol) or downstream renewable syngas separation product (e.g., purified hydrogen), following reaction or separation of the synthesis gas product. A representative process comprises, in a gasifier, contacting the carbonaceous feed with an oxygen-containing gasifier feed, under gasification conditions, to provide a gasifier effluent comprising, as a synthesis gas, H₂, CO, and gasifier effluent tar. This may otherwise be referred to as a tar-containing synthesis gas, a tar-laden synthesis gas, or a raw gasifier effluent. The process may further comprise subjecting the gasifier effluent to a tar removal operation to remove at least a portion of the gasifier effluent tar and provide a tar-depleted gasifier effluent. Advantageously, the temperature and/or residence time of the tar removal operation may be adjusted in response to a measured breakthrough quantity of the gasifier effluent tar.

The breakthrough quantity of tar may be measured quantitatively or qualitatively, (i) directly downstream of the tar removal (e.g., tar reforming) operation, such as by analysis of a tar-depleted gasifier effluent exiting this operation, or (ii) further downstream such as following cooling (e.g., in a quenching operation and/or a CSC). Analysis of the tar-depleted effluent or gasifier effluent that is downstream of this (e.g., an-scrubbed gasifier effluent as described herein) may be performed to determine a level of tar based on one or more components of tar, such as based on an amount (weight percentage or concentration) of benzene, naphthalene, and/or pyrene; based on a combined amount of C₂⁺ hydrocarbons; based on a combined amount of C₆⁺ hydrocarbons and/or C₆⁺ oxygenated hydrocarbons; etc. In this regard, it can be appreciated that any one or more components (specific compounds) known to be present in tar for a given operation can serve as a proxy for the total content of tar and thereby serve as the basis for the temperature and/or residence time adjustment. In the case of qualitative measurement, this may be performed, for example, by detection of condensed tar, or components of condensed tar, on process equipment.

In the case of tar downstream of the tar removal operation exceeding a given threshold (e.g., based on an amount of any tar component or combined amount of two or more components), the severity of the tar removal operation may be increased by increasing temperature and/or residence time of the tar removal operation. According to a preferred embodiment, for example, residence time alone may be varied, such as in the particular case of the tar removal operation utilizing a temperature (e.g., an average temperature or possibly a peak temperature), representing a maximum temperature, not to be exceeded in order to maintain acceptable properties of ash generated in the gasifier. Residence time alone, or optionally in combination with temperature, may therefore represent the variable(s) by which severity of the tar removal operation is adjusted to maintain a given level of performance.

More particularly, temperature and/or residence time may be adjusted to achieve, or adjusted toward (i.e., adjusted in the direction of achieving) a target conversion of tar or target amount (weight percentage or concentration) of tar, relative to a measured conversion or measured amount (weight percentage or concentration), i.e., as an indication of tar breakthrough. That is, the actual measured conversion or measured amount may be calculated or determined based on any determination of tar or one or components of tar (e.g., serving as a proxy for the total content of tar), as described above. For example, a target conversion may be 90%, 95%, 99%, or other representative percentage representing a threshold conversion level. A target amount may be a target weight percentage or target parts per million by weight of 1000 wt-ppm, 100 wt-ppm, 10 wt-ppm, or 1 wt-ppm, or other representative weight percentage representing a threshold amount. Therefore, according to particular embodiments, the severity of the tar removal operation may be increased (e.g., by increasing residence time alone, optionally in combination with increasing temperature) to achieve, or adjust toward, a target conversion that exceeds the measured conversion or otherwise a target amount that is below the measured amount. Conversely, the severity of the tar removal operation may be decreased (e.g., by decreasing residence time alone, optionally in combination with decreasing temperature) to achieve, or adjust toward, a target conversion that is below the measured conversion or otherwise a target amount that exceeds the measured amount.

In view of detrimental effects relating to ash softening and/or slag formation as a result of exposure to high temperatures, residence time adjustment is particularly beneficial in terms of promoting a desired level of performance of the tar removal operation (e.g., based on a measured conversion or measured amount as described above), while minimizing temperature in the overall slate of temperature/residence time combinations that can be used to achieve that performance for a particular operation of the gasifier, processing a particular carbonaceous feed. In a representative embodiment, the temperature of the tar removal operation may be adjusted to a minimum value to achieve the target conversion or target amount, under the overall conditions in the tar removal operation. Such overall conditions include not only this minimum value temperature, but also residence time, pressure, carbonaceous feed composition, and other variables affecting the tar removal operation. This minimum value temperature may be, for example, a minimum average temperature or a minimum peak temperature, with the temperature being measured, for example, throughout a reactor (e.g., a Pox reactor) used in the tar removal operation, or otherwise at one or more discreet points in such reactor. The average temperature or peak temperature of the tar removal operation may also include temperature(s) measured elsewhere in this operation. For example, in some embodiments, the tar removal operation may comprise a tar conversion residence vessel (TCRV) that can facilitate variations in residence time, and optionally the temperature of the tar removal operation (e.g., in determining a minimum value) may be based at least in part on one or more temperatures measured in this vessel.

A TCRV may be positioned directly downstream of a reactor used in the tar removal operation and may be sized for adding a predetermined residence time, i.e., a TCRV-mediated residence time, beyond that of the reactor(s) used in the tar removal operation, for the further destruction of tar and its components through the desired reactions (e.g., reforming and/or oxidation). The TCRV-mediated residence time, for example, may be in the range from about 5 seconds to about 5 minutes, such as from about 10 seconds to about 2 minutes or from about 15 seconds to about 45 seconds. In making adjustments to the residence time of the tar removal operation, as described herein (e.g., in response to a measured breakthrough quantity of gasifier effluent tar), such adjustments may comprise, or consist of (only), adjustments to the TCRV-mediated residence time. These adjustments nonetheless affect the overall residence time, such as the total or combined (i) residence time of the reactor(s) used in the tar removal operation and (ii) TCRV-mediated residence time.

In representative embodiments, adjustment of the residence time of the reactor(s) may be performed at least in part by adjusting the total material flow (e.g., flow of the raw gasifier effluent) through the reactors. Adjustment of the TCRV-mediated residence time may be performed by bypassing the TCRV to a greater or lesser extent. For example, a minimum TCRV-mediated residence (e.g., no TCRV-mediated residence time) may be established by complete bypassing of the TCRV, whereas a maximum TCRV-mediated residence time may be established by complete closing of any bypass around the TCRV such that, for example, the entire effluent of the reactor(s) used in the tar removal operation flows through the TCRV. Partial bypassing can be used to regulate the TCRV-mediated residence time between this minimum and maximum, and therefore the overall residence time of the tar removal operation can be adjusted, or lengthened to the extent allowed by this additional “knob.” Conditions of the tar removal operation may therefore include, as portions of the overall residence time, the residence times (i) and (ii) as noted above, either of both of which may be adjusted, or varied, as described herein to provide added flexibility in effectively achieving the combined objectives of tar removal and management of ash (or the effects of its exposure to high temperatures), preferably without the requirement for a radiant syngas cooler (RSC).

In achieving other various objectives and associated advantages as described herein, other particular embodiments of the invention are directed to processes for gasification of a carbonaceous feed, in order to produce a synthesis gas product and/or optionally a downstream renewable syngas conversion product (e.g., liquid hydrocarbons or methanol) or downstream renewable syngas separation product (e.g., purified hydrogen), following reaction of the synthesis gas product. A representative process comprises, in a gasifier, contacting the carbonaceous feed with an oxygen-containing gasifier feed, under gasification conditions, to provide an un-scrubbed gasifier effluent comprising, as a synthesis gas, H₂, CO, and water-soluble contaminants. These water-soluble contaminants may include poisons (e.g., chlorides, H₂S) of catalysts used for subsequently performing the water-gas shift (WGS) reaction, and/or other undesired byproducts (e.g., NH₃). The process may further comprise feeding at least a portion of the un-scrubbed gasifier effluent to a scrubbing operation to remove at least a portion of the water-soluble contaminants, and provide a scrubbed gasifier effluent.

The scrubbed gasifier effluent typically has a reduced amount (weight percentage or concentration) of the water-soluble contaminants, in addition to a reduced amount (weight percentage or concentration) of water, relative to the corresponding amounts in the un-scrubbed gasifier effluent (e.g., which may be any synthesis gas product downstream of the gasifier and upstream of the scrubbing operation). The process may also comprise feeding at least a portion of the scrubbed gasifier effluent to a WGS operation, to provide a WGS product having a H₂:CO molar ratio that is increased, relative to that of the scrubbed gasifier effluent. Advantageously, such processes may further comprise cross-exchanging heat between at least a portion of the scrubbed gasifier effluent and at least a portion of the un-scrubbed gasifier effluent. Such cross-exchanging may provide effective heat utilization from within the process (e.g., utilization of heat originally generated in the gasifier and/or tar removal operation) to achieve favorable conditions in the synthesis gas that is a feed to the WGS operation, which according to particular embodiments may be a cross-exchanger heated effluent.

In achieving yet other various objectives and associated advantages as described herein, yet other particular embodiments of the invention are directed to processes for gasification of a carbonaceous feed to produce a renewable syngas conversion product (e.g., liquid hydrocarbons or methanol) or a renewable syngas separation product (e.g., purified hydrogen). A representative process comprises, in a gasifier, contacting the carbonaceous feed with an oxygen-containing gasifier feed, under gasification conditions, to provide a gasifier effluent comprising H₂ and CO and gasifier effluent tar; subjecting the gasifier effluent to a tar removal operation to remove at least a portion of the gasifier effluent tar and provide a tar-depleted gasifier effluent; feeding at least a portion of the tar-depleted gasifier effluent, optionally following one more intervening operations downstream of the gasifier, to a WGS operation, to provide a WGS product having a H₂:CO molar ratio that is increased, relative to that of the tar-depleted effluent; and feeding at least a portion of the WGS product to (i) a syngas conversion operation to provide the renewable syngas conversion product, or (ii) a syngas separation operation to provide the renewable syngas separation product. According to representative processes, (a) the syngas conversion operation provides a gaseous conversion byproduct comprising unconverted synthesis gas components (H₂, CO), light hydrocarbons (e.g., CH₄, C₂H₆), and/or other non-condensable gases such as CO₂, or (b) the syngas conversion operation provides a liquid conversion byproduct comprising heavy hydrocarbons (e.g., C₂₀⁺ hydrocarbons that include hydrocarbons having a molecular weight beyond those considered diesel boiling-range hydrocarbons or aviation fuel boiling-range hydrocarbons and/or include hydrocarbons that are solid at room temperature) and/or heavy alcohols (e.g., amyl alcohols that may be present in a fusel oil fraction), or (c) the syngas separation operation provides a gaseous separation byproduct comprising separated synthesis gas components (e.g., a tail gas obtained from pressure swing adsorption (PSA) that is used to generate high purity hydrogen, with the tail gas being enriched in non-hydrogen components of syngas, such as CO, CO₂, H₂O, and possibly methane).

Advantageously, such processes may further comprise combusting all or at least a portion (e.g., a first portion) of (a) the gaseous conversion byproduct of the syngas conversion operation, (b) the liquid conversion byproduct of the syngas conversion operation, or (c) the gaseous separation byproduct of the syngas separation operation, as a fuel for the tar removal operation. For example, combustion of a tail gas obtained from PSA in the generation of high purity hydrogen, or portion thereof, may occur directly within a Pox reactor, such as in the case of being fed to a hot oxygen burner (HOB) used in this reactor. According to particular embodiments, in addition to, or as an alternative to, feeding a first portion of (a), (b), or (c) above as fuel, the process may comprise feeding a second portion of (a), (b), or (c) above to the gasifier. For example, in the case of feeding both first and second portions, the latter or second portion of the gaseous or liquid conversion byproducts (a) or (b), or the latter or second portion of the gaseous separation byproduct (c) may represent an amount beyond the fuel requirement of the HOB. In any event, the direct utilization of such portion(s) beneficially retains carbon within the process (i.e., provides a pathway for carbon recycle), for purposes of combustion and/or improvement of the yield of syngas and consequently its downstream conversion products.

Representative gasification processes described herein are defined by various possible operations, occurring downstream of the gasifier which may include a tar removal operation; operations for cooling, such as a quenching operation and/or a CSC; a filtration operation; cross-exchanging heat; a scrubbing operation; compression; a WGS operation; a sour water treating operation; and a syngas conversion operation. Certain possible features of the gasifier, as well as these downstream operations and their associated process streams and conditions, according to preferred embodiments and otherwise any embodiments as defined in the claims, as well as the embodiment illustrated in the FIGURE, are provided in the following description.

### Gasifier

Representative processes comprise, in a gasifier, contacting a carbonaceous feed with an oxygen-containing gasifier feed, under gasification conditions, to provide a gasifier effluent (e.g., a raw gasifier effluent) comprising synthesis gas.

The carbonaceous feed may comprise coal (e.g., high quality anthracite or bituminous coal, or lesser quality subbituminous, lignite, or peat), petroleum coke, asphaltene, and/or liquid petroleum residue, or other fossil-derived substance. In a preferred embodiment, the carbonaceous feed may comprise biomass. The term “biomass” refers to renewable (non-fossil-derived) substances derived from organisms living above the earth's surface or within the earth's oceans, rivers, and/or lakes. Representative biomass can include any plant material, or mixture of plant materials, such as a hardwood (e.g., whitewood), a softwood, a hardwood or softwood bark, lignin, algae, and/or lemna (sea weeds). Energy crops, or otherwise agricultural residues (e.g., logging residues) or other types of plant wastes or plant-derived wastes, may also be used as plant materials. Specific exemplary plant materials include corn fiber, corn stover, and sugar cane bagasse, in addition to “on-purpose” energy crops such as switchgrass, miscanthus, and algae. Short rotation forestry products, such as energy crops, include alder, ash, southern beech, birch, eucalyptus, poplar, willow, paper mulberry, Australian Blackwood, sycamore, and varieties of paulownia elongate. Other examples of suitable biomass include vegetable oils, carbohydrates (e.g., sugars), organic waste materials, such as waste paper, construction, demolition wastes, digester sludge, and biosludge. Representative carbonaceous feeds therefore include, or comprise, any of these types of biomass. Particular carbonaceous feeds comprising biomass include municipal solid waste (MSW) or products derived from MSW, such as refuse derived fuel (RDF). Carbonaceous feeds may comprise a combination of fossil-derived and renewable substances, including those described above. A preferred carbonaceous feed is wood.

In the gasifier (or, more particularly, a gasification reactor of this gasifier), the carbonaceous feed is subjected to partial oxidation in the presence of an oxygen-containing gasifier feed, added in an amount generally limited to supply only 20-70% of the oxygen that would be necessary for complete combustion. The oxygen-containing gasifier feed will generally comprise other oxygenated gaseous components including H₂O and/or CO₂ that may likewise serve as oxidants of the carbonaceous feed. The oxygen-containing gasifier feed can refer to all gases being fed or added to the gasifier, or otherwise can refer to gas that is separate from other gases being fed or added, whether subsequently combined upstream of, or within, the gasifier. For example, the oxygen-containing gasifier feed may be introduced to the gasifier, along with steam, or a portion of steam, generated elsewhere in the process (e.g., CSC-generated steam) and used as a separate feed. Contacting of the carbonaceous feed with the oxygen-containing gasifier feed in the gasifier provides a gasifier effluent, and more particularly a raw gasifier effluent as the product directly exiting the gasifier. One or more reactors (e.g., in series or parallel) of the gasifier may operate under gasification conditions present in such reactor(s), with these conditions including a temperature of generally from about 500 °C (932 °F) to about 1000 °C (1832 °F), and typically from about 816 °C (1500 °F) to about 1038 °C (1900 °F). Other gasification conditions may include atmospheric pressure or elevated pressure, for example an absolute pressure generally from about 0.1 megapascals (MPa) (14.5 psi) to about 10 MPa (1450 psi), and typically from about 1 MPa (145 psi) to about 3 MPa (435 psi), or from about 0.5 MPa (72 psi) to about 2 MPa (290 psi).

Gasification reactor configurations include counter-current fixed bed (“up draft”), co-current fixed bed (“down draft”), and entrained flow plasma. Different solid catalysts, having differing activities for one or more desired functions in gasification, such as tar reduction, enhanced H₂ yield, and/or reduced CO₂ yield, may be used. Limestone may be added to a gasification reactor, for example, to promote tar reduction by cracking. Various catalytic materials may be used in a gasification reactor, including solid particles of dolomite, supported nickel, alkali metals, and alkali metal compounds such as alkali metal carbonates, bicarbonates, and hydroxides. Often, a gasifier is operated with a gasification reactor having a fluidized bed of particles of the carbonaceous feed (and optionally particles of solid catalyst), with the oxygen-containing gasifier feed, and optionally separate, fluidizing H₂O- and/or CO₂-containing feeds, being fed upwardly through the particle bed. Exemplary types of fluidized beds include bubbling fluidized beds and entrained fluidized beds.

In addition to gasifier effluent tar, the raw gasifier effluent comprises CO, CO₂, and methane (CH₄) that are derived from the carbon present in the carbonaceous feed, as well as H₂ and/or H₂O, and generally both, together with other components in minor concentrations, as described below. According to the embodiment illustrated in the FIGURE, the raw gasifier effluent 16 may be obtained directly from gasifier 50, prior to further operations as described herein.

The raw gasifier effluent, or any gasifier effluent having been subjected to one or more operations as described herein, may comprise synthesis gas, i.e., may comprise both H₂ and CO, with these components being present in various amounts (concentrations), and preferably in a combined amount of greater than about 25 mol-% (e.g., from about 25 mol-% to about 95 mol-%), greater than about 50 mol-% (e.g., from about 50 mol-% to about 90 mol-%), or greater than about 65 mol-% (e.g., from about 65 mol-% to about 85 mol-%). With respect to any such combined amounts (concentrations), the H₂:CO molar ratio of the gasifier effluent may be suitable for use in downstream syngas conversion operations (reactions or separations), such as (i) the conversion to a renewable syngas conversion product comprising higher molecular weight hydrocarbons and/or alcohols of varying carbon numbers via Fischer-Tropsch conversion or (ii) the conversion to a renewable syngas conversion product comprising methanol via a catalytic methanol synthesis reaction, or (iii) the conversion to a renewable syngas conversion product comprising renewable natural gas (RNG) via catalytic methanation that increases the methane content in a resulting RNG stream, or (iv) the separation of a renewable syngas separation product comprising purified hydrogen. More typically, however, a WGS operation is needed to achieve a favorable H₂:CO molar ratio, and/or a favorable H₂ concentration, for these or other downstream syngas conversion and separation operations. For example, the WGS operation may include parameters (e.g., reactor temperatures and/or catalyst types) for obtaining the highest yield/concentration of hydrogen, through consumption of CO present in the syngas upstream of this operation, in the case obtaining purified hydrogen as a renewable syngas separation product (e.g., by utilizing one or more PSA and/or membrane separation stages).

Independently of, or in combination with, the representative amounts (concentrations) of H₂ and CO above, the gasifier effluent may comprise CO₂, for example in an amount of at least about 2 mol-% (e.g., from about 2 mol-% to about 30 mol-%), at least about 5 mol-% (e.g., from about 5 mol-% to about 25 mol-%), or at least about 10 mol-% (e.g., from about 10 mol-% to about 20 mol-%). Independently of, or in combination with, the representative amounts (concentrations) of H₂, CO, and CO₂ above, the gasifier effluent may comprise CH₄, for example in an amount of at least about 0.5 mol-% (e.g., from about 0.5 mol-% to about 15 mol-%), at least about 1 mol-% (e.g., from about 1 mol-% to about 10 mol-%), or at least about 2 mol-% (e.g., from about 2 mol-% to about 8 mol-%). Together with any water vapor (H₂O), these non-condensable gases H₂, CO, CO₂, and CH₄ may account for substantially all of the composition of the gasifier effluent. That is, these non-condensable gases and any water may be present in the gasifier effluent in a combined amount of at least about 90 mol-%, at least about 95 mol-%, or even at least about 99 mol-%.

### Tar Removal Operation

The raw gasifier effluent, obtained directly from the gasifier, will generally comprise gasifier effluent tar, such that a tar removal operation is typically necessary for further processing. This gasifier effluent tar can include compounds that are referred to in the art as “tars” and “oils” and are more particularly hydrocarbons and oxygenated hydrocarbons having molecular weights greater than that of methane, which may be present in the gasifier effluent at concentrations ranging from several wt-ppm to several wt-%. Certain types of these compounds, having relatively high molecular weight, are further characterized by being problematic due to their tendency to condense at lower temperatures and coat internal surfaces of processing equipment, downstream of the gasifier, causing undesirable fouling, corrosion, and/or plugging. These compounds also interfere with subsequent processing steps, or syngas conversion operations, for upgrading synthesis gas to higher value products, which perform optimally (e.g., from the standpoint of stability) with pure feed gases.

Particular compounds that are undesirable for these reasons include hydrocarbons and oxygenated hydrocarbons having six carbon atoms or more (C₆⁺ hydrocarbons and oxygenated hydrocarbons), with benzene, toluene, xylenes, naphthalene, pyrene, phenol, and cresols being specific examples. These compounds are typically present in the raw gasifier effluent in a total (combined) amount from 1-100 g/Nm³. The removal (e.g., by conversion) of these organic compounds is therefore generally necessary to avoid serious problems caused by their deposition over time. Other types of tars and oils, such as ethane, ethylene, and acetylene, will not condense from the gasifier effluent but will nonetheless “tie up” hydrogen and carbon, with the effect of reducing the overall yield of H₂ and CO as the desired components of synthesis gas.

Depending on the specific tar removal operation, tars and oils in the raw gasifier effluent can be converted, either catalytically or non-catalytically, by oxidation, cracking, and/or reforming to provide, in the tar-depleted gasifier effluent, additional H₂ and CO. The tar conversion reaction(s) can utilize available O₂ or oxygen sources (e.g., H₂O and/or CO₂) that are present in, and/or added to, the synthesis gas. In view of the gasifier effluent tar, together with methane, containing a significant portion of the energy of the raw gasifier effluent, the conversion of these compounds can increase the overall yield of synthesis gas substantially. The tar removal operation, which may therefore, according to certain embodiments, be more specifically a tar conversion operation, can effectively reduce the concentration of compounds present as tar in the raw gasifier effluent, having been produced in the gasifier. In general, tar removal, and more particularly tar conversion reactions, may be performed under higher temperatures compared to those used in the gasifier, such that the tar-depleted gasifier effluent, obtained directly from the tar removal operation, may have a temperature of greater than about 1000 °C (e.g., from about 1000 °C (1832 °F) to about 1500 °C (2732 °F), such as from about 1204 °C (2200 °F) to about 1427 °C (2600 °F)).

According to one embodiment, the tar removal operation may be used for the conversion (e.g., reforming) of tar and methane through non-catalytic partial oxidation (Pox) in a reactor used for this operation. The efficiency of this specific operation can be promoted using hot oxygen burner (HOB) technology, according to which an excess of oxygen is mixed with a small amount of fuel (e.g., natural gas, propane, or recycled synthesis gas). Combustion of this fuel within the reactor can result in a temperature increase to above 1100 °C (2012 °F), causing the combustion products and excess oxygen to accelerate to sonic velocity through a nozzle, thereby forming a turbulent jet that enhances mixing between the tar/methane containing synthesis gas and the reactive hot oxygen stream. An HOB-based system can effectively improve synthesis gas yields.

In the case of a tar removal operation that utilizes catalytic conversion of tar and methane, this operation may include a reactor containing a bed of catalyst comprising solid or supported Ni, solid or supported Fe, and/or dolomite, for example in the form of a secondary fluidized bed downstream of the gasifier. Other catalysts for tar conversion include olivine, limestone, zeolites, and even metal-containing char produced from the gasification. As in the case of non-catalytic processes that may be performed in a tar removal operation, catalytic tar conversion may likewise include the introduction of supplemental oxygen and/or steam reactants, into a reactor used for this operation.

According to other particular embodiments, the tar removal operation may utilize a suitable liquid or solid adsorbent, to selectively adsorb tars and oils from the raw gasifier effluent. For example, the tar removal operation may be performed with an oil washing system, whereby the raw gasifier effluent is passed through (contacted with) a liquid medium such as bio-oil liquor, to extract the tars and oils based on their preferential solubility. The liquid adsorbent may be combusted after it has become spent.

Regardless of the particular method by which the tar removal operation is performed, the raw gasifier effluent may comprise tars and oils (e.g., present as compounds described above) in an amount, or combined amount, from about 0.01 wt-% to about 5 wt-%, such as from about 0.1 wt-% to about 3 wt-% or from about 0.5 wt-% to about 2 wt-%. The tar removal operation may be effective to substantially or completely remove this gasifier effluent tar. For example, the tar-depleted gasifier effluent exiting, or obtained directly from, this operation, may comprise tars and oils in an amount, or combined amount, of less than about 0.5 wt-%, less than about 0.1 wt-%, or less than about 0.01 wt-%. Representative levels of removal of tars and oils (e.g., by conversion), measured across the tar removal operation, may be at least about 90%, at least about 95%, or even at least about 99%, resulting in a tar-depleted gasifier effluent that may be substantially or completely free of tar.

### Quenching Operation

Hot gasifier effluent, for example the tar-depleted gasifier effluent exiting the tar removal operation, can be cooled by various techniques that include radiant and/or convective heat exchange. In representative embodiments, at least one quenching operation, and preferably a dry quenching operation, is used, in which water is added directly to the gasifier effluent and contributes to its overall moisture content, thereby favoring H₂ production via the equilibrium-limited WGS reaction (i.e., to provide an increased H₂:CO molar ratio and an increased H₂ concentration). A dry quenching operation utilizes the sensible heat of the gasifier effluent to vaporize the injected water, which is sufficient for obtaining the resulting quenched gasifier effluent at a desired, cooler temperature. In the case of avoiding the use of an RSC according to preferred embodiments, the quenched gasifier effluent may have a temperature from about 400 °C (752 °F) to about 900 °C (1652 °F), and preferably from about 538 °C (1000 °F) to about 816 °C (1500 °F) to allow for further processing. This can include, after sufficient further cooling (e.g., using a CSC) a subsequent filtration operation (passage through a filter) to remove solid particles (e.g., dust). In preferred embodiments, only a partial quench is used in the quenching operation, as opposed to a full quench, such that the quenched gasifier effluent exiting, or obtained directly from, the dry quenching operation is above its dewpoint, i.e., not saturated. In general, the dry quenching operation can promote rapid and efficient cooling through direct contact between hot gasifier effluent and water or other aqueous quenching medium.

### Convective Syngas Cooler (CSC)

As described herein, according to preferred embodiments, a combination of a quenching operation characterized by direct contact of a synthesis gas (e.g., the tar-depleted gasifier effluent exiting the tar removal operation) and a quenching medium such as water, together with a CSC, can provide effective cooling for further downstream operations, without reliance on an RSC for required removal of ash and formed slag. For example, a CSC may be used to cool a quenched gasifier effluent exiting the quenching operation to provide a cooled gasifier effluent, with the quenched gasifier effluent optionally having a temperature within a range as described above and/or the cooled gasifier effluent having temperature from about 250 °C (482 °F) to about 600 °C (1112 °F), and preferably from about 316 °C (600 °F) to about 399 °C (750 °F) to allow for subsequent filtration. A CSC may operate by indirect heat transfer, such as in the case of having a shell and tube configuration, and typically generates steam from some of the heat recovered from the gasifier and tar removal operation. According to more particular embodiments, a CSC operates as a boiler (e.g., a fire tube boiler or water tube boiler) for the production of high and/or intermediate pressure steam.

### Filtration Operation

A filtration operation, using any suitable filter, may be used to remove solid particles (particulates) from the gasifier effluent, for example the cooled gasifier effluent as described above, exiting the CSC. In the case of biomass gasification, these solid particles can include char, tar, soot, and ash, any of which can generally contain alkali metals such as sodium. Corrosive and/or harmful species such as chlorides, arsenic, and/or mercury may also be contained in such solid particles. A high temperature filtration, for example using bundles of metal or ceramic filters, may generally be sufficient to reduce the content of solid particles in the gasifier effluent, such as to provide a filtered gasifier effluent exiting, or obtained directly from, the filtration operation and having less than 1 wt-ppm, and possibly less than 0.1 wt-ppm of solid particles. In representative embodiments, the filtered gasifier effluent may have a temperature in a range as described above with respect to the cooled gasifier effluent.

In some embodiments, a filtration operation may be performed upstream of (prior to) the tar removal operation to allow the latter to operate more effectively. The removal of solid particles of varying average particles sizes, using filtration or other techniques, may be performed at any of a number of possible stages within the overall process. For example, coarse solids removal by centrifugation may be performed directly downstream of the gasifier, and/or may even be performed in situ in the gasifier (e.g., using internal cyclones, for removal of solid particles, positioned in a headspace above a fluidized particle bed).

The filtration operation may be followed by, or integrated with, a supplemental cleaning operation to further purify the gasifier effluent, such as to further reduce its tar and overall hydrocarbon content, for example by contact with a solid “polishing” material such as a carbon bed. This can provide for more thorough removal of benzene, naphthalene, pyrene, toluene, phenols, and other condensable species that could otherwise be detrimental to downstream operations, such as by deposition onto equipment.

### Scrubbing Operation

A scrubbing operation may be used to remove water and water-soluble contaminants from an un-scrubbed gasifier effluent, such as the filtered gasifier effluent exiting the filtration operation, and optionally following the cooling of this stream by cross-exchanging heat. For example, the filtered gasifier effluent may be characterized as a cross-exchanger heated feed that, following the cross-exchanging, provides a cross-exchanger cooled effluent that may be characterized as a feed to the scrubbing operation. The scrubbing operation may provide further cooling of the cross-exchanger cooled effluent. For example, the cross-exchanger cooled effluent, optionally following cooling of the filtered gasifier effluent having a temperature as described above, may have a temperature from about 200 °C (392 °F) to about 450 °C (842 °F), and preferably from about 260 °C (500 °F) to about 371 °C (700 °F), whereas the scrubbed gasifier effluent exiting the scrubber, or optionally a compressed, scrubbed gasifier effluent additionally following compression, may be characterized as a cross-exchanger cooled feed, may have a temperature from about 35 °C (95 °F) to about 100 °C (212°F), and preferably from about 43°C (110 °F) to about 66 °C (150 °F).

The scrubbing operation, such as wet scrubbing, may be effective for removing, as water-soluble contaminants, chlorides (e.g., in the form of HCl), ammonia, and HCN, as well as fine solid particles (e.g., char and ash). For example, in the case of using a wet scrubber, an un-scrubbed gasifier effluent (e.g., the cross-exchanger cooled effluent) may be fed to a trayed column to perform co-current or counter-current contacting with water. Further cooling in this column, such as to a temperature below 100 °C (212 °F) can aid in droplet condensation for improving the contaminant removal effectiveness. The scrubbing operation can be used to provide a scrubbed gasifier effluent exiting, or obtained directly from, this operation and having a combined amount of chloride, ammonia, and solid particles of less than 1 wt-ppm, and possibly less than 0.1 wt-ppm. The scrubbing operation also generally serves to remove water, such that the moisture content of the scrubbed gasifier effluent is reduced, relative to the feed to the scrubbing operation (e.g., the cross-exchanger cooled effluent).

### WGS Operation

The water gas shift (WGS) operation reacts CO present in a scrubbed gasifier effluent, for example a cross-exchanger heated effluent downstream of the scrubbing operation, following cross-exchanging heat and optionally compression, with steam to increase H₂ concentration (as well as CO₂ concentration). In this manner, the cross-exchanger heated effluent may be characterized as a feed to the WGS operation. Advantageously, following the tar removal operation, filtration operation, scrubbing operation, and cross-exchanging heat, the cross-exchanger heated effluent/feed to the WGS operation may have favorable properties for use in this operation, in terms of its temperature and its being free or substantially free of water-soluble contaminants as described above, as well as tars and particulates. For example, the cross-exchanger heated effluent/feed to the WGS operation, following subjecting the scrubbed gasifier effluent to cross-exchanging heat and optionally compression, may have a temperature from about 225 °C (437 °F) to about 475 °C (887 °F), and preferably from about 260 °C (500 °F) to about 399 °C (750 °F), whereas the scrubbed gasifier effluent exiting the scrubber, may have a temperature as described above.

In the WGS operation, the use of steam in excess of the stoichiometric amount may be beneficial, particularly in adiabatic, fixed-bed reactors, for a number of purposes. These include driving the equilibrium toward hydrogen production, adding heat capacity to limit the exothermic temperature rise, and minimizing side reactions, such as methanation. In this regard, a supplemental source of steam, adding to that present in the feed to the WGS operation, may be combined with this feed. The supplemental source of steam may be readily available through generation in the process, or it may be external to the process. In a preferred embodiment, at least a portion of steam (e.g., high or medium pressure steam) generated in the CSC may be fed or added to the WGS operation (e.g., to one or more reactors used in this operation), thereby improving overall heat balancing/integration.

Reactors used in a WGS operation may contain a suitable catalyst, such as those comprising one or more of Co, Ni, Mo, and W on a solid support, particular examples of which are Co/Mo and Ni/Mo catalysts that exhibit sulfur tolerance. Other catalysts for use in this operation (i.e., contained within one or more WGS reactors) include those based on copper-containing and/or zinc-containing catalysts, such as Cu—Zn—Al; chromium-containing catalysts; iron oxides; zinc ferrite; magnetite; chromium oxides; and any combination thereof (e.g., Fe₂O₃—Cr₂O₃ catalysts).

In a typical WGS operation, two or more reactors with interstage cooling are used in view of the thermodynamic characteristics of the WGS reaction. For example, a high-temperature shift (HTS) reactor may operate with a temperature of the reactor inlet from about 310 °C (590 °F) to about 450 °C (842 °F), with more favorable reaction kinetics but a less favorable equilibrium conversion. The effluent from the HTS may then be cooled to a temperature suitable for the reactor inlet of a low-temperature shift (LTS) reactor, such as from about 200 °C (392 °F) to about 250 °C (482 °F), for providing less favorable reaction kinetics but a more favorable equilibrium conversion, such that the combined effect of the HTS and LTS reactors results in a high conversion to H₂ with a favorable residence time. In some cases, it may be desirable to use three or more reactors, or catalyst beds, to perform the WGS reaction, again with cooling between consecutive reactors or catalyst beds.

In this manner, the WGS operation may be used to provide an immediate WGS product exiting, or obtained directly from, this operation and having an increased H₂:CO molar ratio and increased H₂ concentration, relative to the feed to the WGS operation (e.g., the cross-exchanger heated effluent), or the synthesis gas obtained from upstream operations (e.g., filtered gasifier effluent or cooled gasifier effluent). For example, the immediate WGS product may have an H₂:CO molar ratio from about 0.5 to about 3.5, from about 1.0 to about 3.0, or from about 1.5 to about 2.5 and/or a hydrogen concentration of at least about 35 mol-% (e.g., from about 35 mol-% to about 80 mol-%), at least about 40 mol-% (e.g., from about 40 mol-% to about 70 mol-%), or at least about 45 mol-% (e.g., from about 45 mol-% to about 65 mol-%). These characteristics of the immediate WGS product may be controlled by bypassing the WGS operation to a greater or lesser extent (e.g., diverting a smaller or larger portion of the feed to this operation, around this operation to provide a portion of the immediate WGS product). The WGS operation may be further beneficial in terms of converting carbonyl sulfide (COS) to H₂S which can be recycled and more easily removed elsewhere in the process, such as in an acid gas removal operation or possibly, at least to some extent, in the scrubbing operation.

### Sour Water Treating Operation

The sour water treating operation is used to remove contaminants such as H₂S and NH₃ present in a sour water byproduct of a scrubbing operation that utilizes an aqueous scrubbing medium. Typically, a combination of heating and steam stripping of the sour water byproduct are used to provide treated water that is substantially free of these contaminants and a condensate enriched in these contaminants, which can be sent for their recovery (e.g., to a sulfur recovery unit to recover H₂S). In this regard, other inventive aspects relate to the use or integration of treated water from a sour water treating operation in a gasification process, such as described herein. For example, the treated water originating from the scrubbing operation may provide all or at least a portion of quench water for a quenching operation used in the process. In this manner, it is possible to include, in a gasification process, a water recovery “loop” (or recycle water loop) comprising the quench water that is input to the quenching operation, any intervening operations between the quenching operation and the scrubbing operation (e.g., the CSC 65, the filtration operation 70, and the gasifier effluent cross-exchanger 75, as illustrated in the FIGURE), a sour water byproduct of the scrubbing operation, and the treated water that is provided by a sour water treating operation. The ability to recover and recycle quench water thereby improves process economics.

### Syngas Conversion or Separation Operations

In some embodiments, processes described herein may also include a syngas conversion operation or syngas separation operation to produce a respective renewable syngas conversion product or renewable syngas separation product, such as liquid hydrocarbons, methanol, or RNG as examples of conversion products, and purified hydrogen as an example of a separation product. In the case of liquid hydrocarbon production, the syngas conversion operation may comprise a Fischer-Tropsch (FT) reaction stage. One or more reactors in this stage are used to process the synthesis gas mixture of hydrogen (H₂) and carbon monoxide (CO) by successive cleavage of C—O bonds and formation of C—C bonds with the incorporation of hydrogen. This mechanism provides for the formation of hydrocarbons, and particularly straight-chain alkanes, with a distribution of molecular weights that can be controlled to some extent by varying the FT reaction conditions and catalyst properties. Such properties include pore size and other characteristics of the support material. The choice of FT catalyst and its active metals (e.g., Fe or Ru) can impact FT product yields in other respects, such as in the production of oxygenates.

In the case of methanol production, the syngas conversion operation may comprise a methanol synthesis reaction stage. One or more reactors in this stage are used to form methanol according to the catalytic reaction:

CO+2H₂→CH₃OH (1 )

Representative catalysts for the synthesis of methanol by this route are characterized by “CZA,” which is a reference to copper and zinc on alumina, or Cu/ZnO/Al₂O₃. Alternatively, or in combination, various other catalytic metals and their oxides may be used, including one or more of W, Zr, In, Pd, Ti, Co, Ga, Ni, Ce, Au, Mn, and their combinations.

In the case of methane production as a syngas conversion operation to provide a renewable natural gas (RNG) product, one or more methanation reactors (e.g., in series or parallel) may be used to react CO and/or CO₂ with hydrogen and thereby provide a hot methanation product having a significantly higher concentration of methane relative to that initially present (e.g., in the WGS product). Catalysts suitable for use in a methanation reactor include supported metals such as ruthenium and/or other noble metals, as well as molybdenum and tungsten. Generally, however, supported nickel catalysts are most cost effective. Often, a methanation reactor is operated using a fixed bed of the catalyst.

In the case of purified hydrogen production, the syngas separation operation may comprise a renewable hydrogen separation stage that can utilize, for example, (i) an adsorbent in the case of separation by PSA or (ii) a membrane. Combinations of such stages may be used in a given syngas separation operation. In any such operation, a gaseous separation byproduct is also provided that is generally enriched in the non-hydrogen components of syngas, such as CO, CO₂, and/or H₂O. This byproduct may be, for example, a PSA tail gas or otherwise a membrane permeate or retentate, depending on the particular membrane used and consequently whether the renewable hydrogen separation product is recovered as the membrane retentate or permeate. This hydrogen, obtained as a result of utilizing a syngas separation operation downstream of the WGS operation, may, in some embodiments, be characterized as high purity hydrogen (e.g., having a purity of at least about 99 mol-% or more, such as at least 99.9 mol-% or at least 99.99 mol-%).

Regardless of the particular syngas conversion operation and/or syngas separation operation used for upgrading synthesis gas such as a WGS product, such operation(s) will generally co-produce a conversion byproduct or separation byproduct (e.g., a gaseous or liquid conversion byproduct or a gaseous separation byproduct) as described herein. For example, a Fischer-Tropsch reaction stage that provides a renewable syngas conversion product comprising liquid hydrocarbons and/or oxygenates may also provide (a) a gaseous conversion byproduct comprising unconverted synthesis gas components (H₂, CO), light hydrocarbons (e.g., CH₄, C₂H₆), and/or other non-condensable gases such as CO₂, and/or (b) a liquid conversion byproduct comprising heavy hydrocarbons (e.g., C₂₀⁺ hydrocarbons that include hydrocarbons having a molecular weight beyond those considered diesel boiling-range hydrocarbons or aviation fuel boiling-range hydrocarbons and/or include hydrocarbons that are solid at room temperature) and/or heavy alcohols. A methanol synthesis reaction stage that provides a renewable syngas conversion product comprising methanol may also provide (a) a gaseous conversion byproduct comprising unconverted synthesis gas components (H₂, CO), light hydrocarbons (e.g., CH₄, C₂H₆), and/or other non-condensable gases such as CO₂, and/or (b) a liquid conversion byproduct comprising heavy alcohols (e.g., amyl alcohols that may be present in a fusel oil fraction). A methanation reaction stage that provides a renewable syngas conversion product comprising RNG may also provide a gaseous conversion byproduct comprising unconverted synthesis gas components (H₂, CO), light hydrocarbons (e.g., CH₄, C₂H₆), and/or other non-condensable gases such as CO₂. A renewable hydrogen separation stage that provides a renewable syngas separation product that is, or comprises, purified hydrogen may also provide a gaseous separation byproduct that is enriched in the non-hydrogen components of syngas, such as CO, CO₂, and/or H₂O. These non-hydrogen components may be present in the gaseous separation byproduct (e.g., a PSA tail gas or otherwise a membrane permeate or retentate) in a combined concentration, for example, of at least about 80 mol-%, at least about 90 mol-%, or at least about 95 mol-%. As described herein, all or portions of these conversion and/or separation byproducts can be advantageously integrated into the overall process to provide certain advantages as described herein.

### Further Exemplary Embodiments of Gasification Processes

The FIGURE depicts a flowscheme illustrating an embodiment of a process including operations as described above, and further integrated with cross-exchanging heat, in addition to the generation and recovery of steam, treated water, and a conversion and/or separation byproduct (e.g., a PSA tail gas). According to this embodiment, in gasifier 50, carbonaceous feed 10 is combined with oxygen-containing gasifier feed 14 under gasification conditions to provide a gasifier effluent, in this case raw gasifier effluent 16 comprising synthesis gas. Oxygen-containing gasifier feed 14 is introduced to gasifier 50, optionally together with a source of steam, which may be first portion 23a of CSC-generated steam 23. Oxygen-containing gasifier feed 14 alone, or possibly in combination with such source of steam, may comprise H₂O and O₂, as well as optionally CO₂, in a combined concentration of at least about 90 mol-%, at least about 95 mol-%, or at least about 99 mol-%.

Raw gasifier effluent 16 is fed to tar removal operation 55, optionally including tar conversion residence time vessel (TCRV) 55a as described herein, for variation of residence time in this operation. This provides tar-depleted gasifier effluent 18, having a lower amount of tar relative to raw gasifier effluent 16. Generally, processes comprise recovering a synthesis gas product from tar-depleted gasifier effluent 16, with such synthesis gas product possibly including any of those downstream of tar-depleted gasifier effluent 16 as illustrated in the FIGURE. For example, the synthesis gas product may be recovered as water-gas shift (WGS) product 36 of WGS operation 90, optionally following one or more intervening operations performed on the gasifier effluent, downstream of the tar removal operation and upstream of the WGS operation. Such intervening operations can include one or more of (i) quenching operation 60 comprising direct contact of the gasifier effluent with quench water 20, (ii) convective syngas cooler (CSC) 65 implementing heat-exchanging contact of the gasifier effluent with boiler feed water 25, (iii) filtration operation 70 to remove solid particles from the gasifier effluent, (iv) scrubbing operation 80 to remove water-soluble contaminants from the gasifier effluent, and (v) cross-exchanging heat between at least a portion of material input to the scrubbing operation (e.g., as cross-exchanger heated feed/filtered gasifier effluent 26) and at least a portion of material removed from the scrubbing operation (e.g., as scrubbed gasifier effluent 30).

Representative processes may further comprise feeding at least a portion of WGS product 36 to syngas conversion operation 95 or syngas separation operation 95 to provide respective renewable syngas conversion product 95 or renewable syngas separation product 40. According to more specific embodiments, for example, (i) syngas conversion operation 95 may comprise a Fischer-Tropsch reaction stage, such that renewable syngas conversion product 40 comprises liquid hydrocarbons and/or oxygenates (e.g., alcohols) of varying carbon numbers, (ii) syngas conversion operation 95 may comprise a catalytic methanol synthesis reaction stage, such that renewable syngas conversion product 40 comprises methanol, or (iii) syngas conversion operation 95 may comprise a catalytic methanation reaction stage, such that renewable syngas conversion product 40 comprises RNG. According to other more specific embodiments, syngas separation operation 95 may comprise a renewable hydrogen separation stage, such that renewable syngas separation product 40 comprises purified hydrogen. Typically, syngas conversion operation provides conversion byproduct 37 or separation byproduct 37 as described herein (e.g., comprising unconverted synthesis gas components, light hydrocarbons, heavy hydrocarbons, and/or fusel oil), and the process may further comprise recycling at least a portion of such byproduct(s). For example, portions of byproduct(s) 37 described herein may be utilized in different operations, such as in the case of first portion 37a being fed to tar removal operation 55 (e.g., as fuel for direct combustion in a hot oxygen burner (HOB) of Pox reactor of this operation) and/or second portion 37b being fed to gasifier 50 (for additional syngas production). In more particular embodiments, second portion 37b may represent an amount of conversion byproduct 37 or separation byproduct beyond a fuel requirement of tar removal operation 55 (e.g., for an HOB used in this operation).

As more particularly illustrated in the FIGURE, a representative process comprises, in quenching operation 60, which may be more particularly a partial dry quench (PDQ) operation, contacting (e.g., by direct contact), tar-depleted gasifier effluent 18 with quench water 20. This provides quenched gasifier effluent 22, having a temperature that is decreased relative to that of tar-depleted gasifier effluent 18. The process may additionally comprise, in convective syngas cooler (CSC) 65, further cooling quenched gasifier effluent 22, such as by indirect, heat-exchanging contact with boiler feed water 25. This provides cooled gasifier effluent 24 and CSC-generated steam 23. In this case, all or at least first portion 23a of CSC-generated steam 23 may be fed to gasifier 50, such as to satisfy its total steam demand according to preferred embodiments, meaning that no supplemental source of steam is required for gasification. In more particular embodiments, in addition to, or alternatively to, first portion 23a being utilized for gasification, second portion 23b of CSC-generated steam 23 (e.g., representing an amount of this total steam that is in excess of that demanded in the gasifier) may be fed to water-gas shift (WGS) operation 90. This operation may also be fed by at least a portion of cooled gasifier effluent 24, optionally following one or more operations to which this stream is subjected, which may be any of those operations specifically illustrated in the FIGURE, including filtration operation 70, cross-exchanging heat with gasifier effluent cross-exchanger 75 (both as a heated feed and a cooled feed), scrubbing operation 80, and compression with compressor 85. Feeding of cooled gasifier effluent 24 to WGS operation 90 provides WGS product 36 having a H₂:CO molar ratio that is increased relative to that of cooled gasifier effluent 24 and/or syngas exiting any of intervening operations, such as filtered gasifier effluent 26 exiting filtration operation 70 or scrubbed gasifier effluent 30 exiting scrubbing operation 80.

As noted, inventive aspects relate to advantages obtained with respect to processes comprising cross-exchanging heat between at least a portion of a scrubbed gasifier effluent, i.e., a synthesis gas downstream of a scrubbing operation as described herein, and at least a portion of an un-scrubbed gasifier effluent, i.e., a synthesis gas upstream of the scrubbing operation as described herein. These advantages are more particularly associated with embodiments in which the scrubbing operation is used upstream of the WGS operation for water-soluble contaminant removal. According to the embodiment illustrated in the FIGURE, for example, the un-scrubbed gasifier effluent may be filtered gasifier effluent 26, having been subjected to filtration operation 70 to remove solid particles. Heat from this un-scrubbed gasifier effluent, which may alternatively be referred to as cross-exchanger heated feed 26, may be cross-exchanged, in gasifier effluent cross-exchanger 75, against scrubbed gasifier effluent 30 exiting scrubbing operation or optionally against compressed, scrubbed gasifier effluent 32, obtained downstream of compressor 85. In this regard, either scrubbed gasifier effluent 30 or compressed, scrubbed gasifier effluent 32 may alternatively be referred to as cross-exchanger cooled feed 30, 32.

An un-scrubbed gasifier effluent, which is subjected to heat exchange against a scrubbed gasifier effluent, may be subjected to various intervening operations, including those illustrated in the FIGURE, between the gasifier and this cross-exchanging of heat. For example, in the case of the un-scrubbed gasifier effluent being filtered gasifier effluent 26, this stream, in addition to having been subjected to filtration operation 70, may have been further subjected (e.g., upstream of this operation) to one or more of (i) tar removal operation 55 to remove at least a portion of the gasifier effluent tar, (ii) quenching operation 60 comprising direct contact with quench water, and (iii) convective syngas cooler (CSC) 65 implementing heat-exchanging contact with boiler feed water. For example (i), (ii), and/or (iii) may be considered intervening operations, and, if used in combination, are preferably performed in the order listed, such as in the order from upstream to downstream of (i), (ii), and (iii).

The operation of cross-exchanging heat, for example occurring in gasifier effluent cross-exchanger 75 as illustrated in the FIGURE, may involve particular steps of (a) cooling a cross-exchanger heated feed (e.g., filtered gasifier effluent 26) to provide cross-exchanger cooled effluent 28 (which may alternatively be referred to as a feed to scrubbing operation 80), with preferably both the cross-exchanger heated feed and cross-exchanger cooled effluent being, or comprising, an un-scrubbed gasifier effluent, i.e., a synthesis gas upstream of the scrubbing operation as described herein, and (b) heating a cross-exchanger cooled feed (e.g., scrubbed gasifier effluent 30 or compressed, scrubbed gasifier effluent 32) to provide cross-exchanger heated effluent 34 (which may alternatively be referred to as a feed to WGS operation 90), with preferably both the cross-exchanger cooled feed and cross-exchanger heated effluent being, or comprising, a scrubbed gasifier effluent, i.e., a synthesis gas downstream of the scrubbing operation as described herein. In the specific case of gasifier effluent cross-exchanger 75 being configured as a shell and tube heat exchanger, the cross-exchanger heated feed and the cross-exchanger cooled effluent may be passed through one side, either the shell side or the tube side, and the cross-exchanger cooled feed and the cross-exchanger heated effluent may be passed through the other side, either the respective tube side or the shell side. In this manner, the use of gasifier effluent cross-exchanger 75 can effectively promote objectives of providing a syngas feed to the WGS that is scrubbed of water-soluble contaminants and heated to a sufficient temperature (e.g., in a range as described above with respect to the cross-exchanger heated effluent/feed to the WGS operation) utilizing available heat from within the process (e.g., heat from the gasifier and/or tar removal operation). In some cases, the use of supplemental heat for heating the scrubbed gasifier effluent upstream of the WGS operation may be avoided, by virtue of cross-exchanging heat.

As further illustrated in the FIGURE, in addition to scrubbed gasifier effluent 30, scrubbing operation 80 additionally provides sour water byproduct 19. Representative gasification processes comprise feeding this byproduct to sour water treating operation 65 to provide treated water 21b, which can be advantageously used as a source of process water. For example, treated water 21b may be sufficient to supply quench water 20 used in quenching operation 60, or possibly at least a portion of quench water, with another portion being supplied by makeup quench water 21a.

Overall, aspects of the invention relate to gasification processes implementing one or a combination of strategies as described herein, such as residence time variation, integration of generated steam, cross-exchanging heat, utilization of gaseous and/or liquid byproducts of conversion and/or separation operations, and recycle of treated water, in the production of synthesis gas or its downstream conversion products (e.g., hydrocarbons, methanol or other alcohols, RNG, or hydrogen), with such strategies potentially leading to improved processing flexibility and/or economics. Specific advantages can include, for example, (i) a reduction in capital cost (e.g., by about 10% or more) in the case of eliminating a radiant syngas cooler (RSC) in exchange for one or more of a TCRV, PDQ, CSC, and a gasifier effluent cross-exchanger, (ii) an increase in the H₂:CO molar ratio of synthesis gas downstream of the tar removal operation, made possible by the TCRV to facilitate lower operating temperatures of this operation (e.g., in a Pox reactor), (iii) improved heat integration, upstream of the WGS operation, and/or (iv) improved syngas yield via utilization of gaseous and/or liquid byproduct recycle for direct fuel combustion or conversion within the process (e.g., within the Pox reactor or within the gasifier).

Those skilled in the art, having knowledge of the present disclosure, will recognize that various changes can be made to these processes in attaining these and other advantages, without departing from the scope of the present disclosure. As such, it should be understood that the features of the disclosure are susceptible to modifications and/or substitutions, and the specific embodiments illustrated and described herein are for illustrative purposes only, and not limiting of the invention as set forth in the appended claims.

1. A process for gasification of a carbonaceous feed, the process comprising:
in a gasifier, contacting the carbonaceous feed with an oxygen-containing gasifier feed, under gasification conditions, to provide a gasifier effluent comprising H₂, CO, and gasifier effluent tar;
subjecting the gasifier effluent to a tar removal operation to remove at least a portion of the gasifier effluent tar and provide a tar-depleted gasifier effluent;
wherein a temperature and/or a residence time of the tar removal operation are adjusted in response to a measured breakthrough quantity of said gasifier effluent tar.

2. The process of claim 1, wherein said temperature and/or residence time are adjusted toward achieving a target conversion of tar or target concentration of tar.

3. The process of claim 2, wherein:
said temperature of the tar removal operation is adjusted to a minimum value, based on said target conversion or target concentration, under conditions in said tar removal operation,
said tar removal operation comprises a tar conversion residence vessel (TCRV), and
said conditions include a TCRV-mediated residence time.

4. The process of claim 1, further comprising:
in a partial dry quench (PDQ) operation, contacting the tar-depleted gasifier effluent with quench water to provide a quenched gasifier effluent; and
in a convective syngas cooler (CSC), further cooling the quenched gasifier effluent to provide a cooled gasifier effluent and CSC-generated steam.

5. The process of claim 4, further comprising feeding all or at least a first portion of the CSC-generated steam to the gasifier.

6. The process of claim 5, comprising feeding the first portion of the CSC-generated steam to the gasifier, the process further comprising feeding a second portion of the CSC-generated steam to a water-gas shift (WGS) operation, fed by at least a portion of the cooled gasifier effluent, to provide a WGS product having a H₂:CO molar ratio that is increased, relative to that of the cooled gasifier effluent.

7. The process of claim 1, further comprising recovering a synthesis gas product from the tar-depleted gasifier effluent.

8. The process of claim 7, wherein the synthesis gas product is recovered as a water-gas shift (WGS) product of a WGS operation, optionally following one or more intervening operations performed on the gasifier effluent, downstream of the tar removal operation and upstream of the WGS operation.

9. The process of claim 8, wherein the one or more intervening operations include one or more of (i) a quenching operation comprising direct contact with quench water, (ii) a convective syngas cooler (CSC) implementing heat-exchanging contact with boiler feed water, (iii) a filtration operation to remove solid particles, (iv) a scrubbing operation to remove water-soluble contaminants, and (v) cross-exchanging heat between at least a portion of material input to the scrubbing operation and at least a portion of material removed from the scrubbing operation.

10. The process of claim 8, further comprising feeding at least a portion of the WGS product to a syngas conversion operation to provide a renewable liquid conversion product.

11. The process of claim 10, wherein the syngas conversion operation (i) comprises a Fischer-Tropsch reaction stage and the renewable liquid conversion product comprises liquid hydrocarbons, or (ii) a methanol synthesis reaction stage and the renewable liquid conversion product comprises methanol.

12. The process of claim 10, wherein the synthesis gas conversion operation provides a gaseous conversion byproduct comprising unconverted synthesis gas components and/or light hydrocarbons, or a liquid conversion byproduct comprising heavy hydrocarbons and/or heavy alcohols, and wherein the process further comprises recycling at least a portion of the gaseous conversion byproduct or the liquid conversion byproduct.

13. A process for gasification of a carbonaceous feed, the process comprising:
in a gasifier, contacting the carbonaceous feed with an oxygen-containing gasifier feed, under gasification conditions, to provide an un-scrubbed gasifier effluent comprising H₂, CO, and water-soluble contaminants;
feeding at least a portion of the un-scrubbed gasifier effluent to a scrubbing operation to remove at least a portion of the water-soluble contaminants, and provide a scrubbed gasifier effluent; and
feeding at least a portion of the scrubbed gasifier effluent to a water-gas shift (WGS) operation, to provide a WGS product having a H₂:CO molar ratio that is increased, relative to that of the scrubbed gasifier effluent,
wherein the process further comprises cross-exchanging heat between at least a portion of the scrubbed gasifier effluent and at least a portion of the un-scrubbed gasifier effluent.

14. The process of claim 13, wherein said cross-exchanging heat occurs in a gasifier effluent cross-exchanger, performing steps of:
cooling a cross-exchanger heated feed to provide a cross-exchanger cooled effluent, wherein the cross-exchanger heated feed and cross-exchanger cooled effluent comprise the un-scrubbed gasifier effluent; and
heating a cross-exchanger cooled feed to provide a cross-exchanger heated effluent, wherein the cross-exchanger cooled feed and cross-exchanger heated effluent comprise the scrubbed gasifier effluent.

15. The process of claim 14, wherein said un-scrubbed gasifier effluent is a filtered gasifier effluent, having been subjected to a filtration operation to remove solid particles.

16. (canceled)

17. The process of claim 13, wherein, in addition to the scrubbed gasifier effluent, the scrubbing operation further provides a sour water byproduct, the process further comprising:
feeding the sour water byproduct to a sour water treating operation, to provide treated water, and
utilizing at least a portion of the treated water in the quenching operation.

18. A process for gasification of a carbonaceous feed to produce a renewable syngas conversion product or a renewable syngas separation product, the process comprising:
in a gasifier, contacting the carbonaceous feed with an oxygen-containing gasifier feed, under gasification conditions, to provide a gasifier effluent comprising H₂ and CO and gasifier effluent tar;
subjecting the gasifier effluent to a tar removal operation to remove at least a portion of the gasifier effluent tar and provide a tar-depleted gasifier effluent;
feeding at least a portion of the tar-depleted gasifier effluent, optionally following one more intervening operations downstream of the gasifier, to a water-gas shift (WGS) operation, to provide a WGS product having a H₂:CO molar ratio that is increased, relative to that of the tar-depleted effluent;
feeding at least a portion of the WGS product to (i) a syngas conversion operation to provide the renewable syngas conversion product, or (ii) a syngas separation operation to provide the renewable syngas separation product;
wherein (a) the syngas conversion operation provides a gaseous conversion byproduct comprising unconverted synthesis gas components and/or light hydrocarbons, and the process further comprises combusting all or at least a first portion of the gaseous conversion byproduct as fuel for the tar removal operation,
(b) the syngas conversion operation provides a liquid conversion byproduct comprising heavy hydrocarbons and/or alcohols and the process further comprises combusting all or at least a first portion of the liquid conversion byproduct as fuel for the tar removal operation, or
(c) the syngas separation operation provides a gaseous separation byproduct comprising separated synthesis gas components and the process further comprises combusting all or at least a first portion of the gaseous separation byproduct as fuel for the tar removal operation.

19. The process of claim 18, comprising:
(a) feeding the first portion of the gaseous conversion byproduct as fuel for the tar removal operation, wherein the process further comprises feeding a second portion of the gaseous conversion byproduct to the gasifier;
(b) feeding the first portion of the liquid conversion byproduct as fuel for the tar removal operation, wherein the process further comprises feeding a second portion of the liquid conversion byproduct to the gasifier; or
(c) feeding the first portion of the gaseous separation byproduct as a fuel for the tar removal operation, wherein the process further comprises feeding a second portion of the gaseous separation product to the gasifier.

20. The process of claim 18, wherein the syngas conversion operation comprises (i) a Fischer-Tropsch reaction stage and the renewable syngas conversion product comprises liquid hydrocarbons, (ii) a methanol synthesis reaction stage and the renewable syngas conversion product comprises methanol, or (iii) a methanation reaction stage and the renewable syngas conversion product comprises renewable natural gas (RNG).

21. The process of claim 18, wherein the syngas separation operation comprises a renewable hydrogen separation stage and the renewable syngas separation product comprises purified hydrogen.
